(19) United States
(12) Patent Application Publication
(10) Pub. No.: US 2021/0369769 A1
(43) Pub. Date: Dec. 2, 2021

(54) MANAGEMENT OF RISK OF CATION OVERLOAD AND ELECTROLYTE IMBALANCE WITH TOPICALLY APPLIED BUFFERS

(71) Applicant: AMPERSAND BIOPHARMACEUTICALS, INC., Thousand Oaks, CA (US)

(72) Inventors: Ryan Beal, Thousand Oaks, CA (US); Luke Gonzales, Thousand Oaks, CA (US); Kilmar Martinez, Thousand Oaks, CA (US); Brandon Sand, Thousand Oaks, CA (US); Lisa Misell, Thousand Oaks, CA (US); Nathan Fitzsimmons, Thousand Oaks, CA (US)

(73) Assignee: DYVE BIOSCIENCES, INC., Thousand Oaks, CA (US)

(21) Appl. No.: 17/290,942

(22) PCT Filed: Nov. 6, 2019
(86) PCT No.: PCT/US2019/060156

§ 371 Date: May 3, 2021

Related U.S. Application Data
(60) Provisional application No. 62/755,388, filed on Nov. 2, 2018.

(51) Int. Cl. A61K 33/10; A61K 9/00; A61K 31/198; A61K 31/133; A61K 31/4172; A61K 47/10; A61K 47/44; A61K 47/34; A61K 47/02; A61K 33/00

(52) U.S. Cl. CPC A61K 33/10; A61K 9/0014; A61K 31/198; A61K 31/133; A61K 33/00; A61K 47/10; A61K 47/44; A61K 47/34; A61K 47/02; A61K 31/4172

(57) ABSTRACT

Provided herein are formulations for the safe and effective topical delivery of buffering agents. The invention includes formulations and methods to balance electrolytes, overcome cation overload, and/or deliver buffers with and without counterions that can be combined in a single use formulation or alternatively in separately applied formulations. Also provided are methods of using the formulations for the treatment of a wide variety of disorders relating to electrolyte imbalance, cation overload, or related conditions.

### CROSS-REFERENCE TO RELATED APPLICATIONS

This application is related to and claims priority to U.S. Provisional Application Ser. No. 62/755, 388 filed Nov. 2, 2018, and is incorporated by reference in its entirety herein.

### FIELD OF INVENTION

This invention relates generally to topical formulations comprising therapeutic agents, and in particular formulations and methods for counteracting or treating unwanted cation overload and electrolyte imbalances and associated conditions and disorders.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

It has previously been shown in Applicant's recent related patent filings that multiple disease states create an acidic extracellular environment due to increased glycolysis in situations of biologic stress. Among our previously disclosed examples is the highly acidic tumor microenvironment observed in cancer. In cancer, this acidic tumor microenvironment has been shown to be a critical influencer of a tumor's metastatic fate and ultimately to the survival of the subject.

Importantly, buffer therapy whether applied orally, intraperitoneally, or topically has been shown to effectively modulate this acidic microenvironment and significant decrease metastases and increase survival. In addition, buffer therapy has been shown to synergistically improve the efficacy of a number of chemotherapeutics and immunotherapies.

One approach that has demonstrated significant promise is topically delivered buffering therapies as described in U.S. application Ser. No. 16/132,358 filed Sep. 14, 2018, entitled ‘Methods and Formulations For Transdermal Administration Of Buffering Agents’, and International Patent Application No. PCT/US18/51250 filed Sep. 14, 2018, entitled ‘Methods of Administration and Treatment’, both incorporated by reference in their entirety herein. This approach avoids the bioavailability and adherence challenges observed in orally and parenterally delivered buffer therapy. These formulations and methods, however, may introduce unwanted cation overload and electrolyte imbalances if delivered chronically or at high doses.

Electrolytes are chemicals in the body that regulate important physiological functions. Examples of electrolytes are sodium, chloride, magnesium, potassium and calcium. Electrolyte imbalance causes a variety of symptoms that can be severe. Electrolyte imbalance is commonly caused by loss of body fluids through prolonged vomiting, diarrhea, sweating or high fever. The most serious forms of electrolyte imbalance in cancer patients include high blood calcium levels, called hypercalcemia, or a disorder called tumor lysis syndrome that results in electrolyte imbalance from the killing of cancer cells. Both of these can be life-threatening if not managed appropriately.

Attempts have been made to mitigate the electrolyte imbalance caused by oral administration of buffering agents by using buffering agents in combination. However, orally delivered buffer therapy has been wrought with bioavailability and adherence challenges that undermine its therapeutic potential for many applications. Formulations suitable for topical delivery for the treatment of cation overload and electrolyte imbalance have not been previously examined for the potential to be used in conjunction with other topically administered formulations comprising buffering agents.

There is a need for formulations and compositions that can be administered in conjunction with the topical delivery of buffering agents and therapies such as those described above for counteracting or treating unwanted cation overload and electrolyte imbalances and the side effects associated with them. The formulations and methods provided herein satisfy this need.

### SUMMARY

The inventions described and claimed herein have many attributes and embodiments including, but not limited to, those set forth or described or referenced in this Brief Summary The inventions described and claimed herein are not limited to, or by, the features or embodiments identified in this Summary, which is included for purposes of illustration only and not restriction.

Multiple disease states create an acidic extracellular environment due to increased glycolysis during situations of biological stress. Applicants have previously shown that buffering therapy can be an effective treatment for some diseases and disorders, and in particular using sodium bicarbonate as a buffering agent.

For metabolic states that generate a significant acid load, oral buffers are unable to delivery an effective dose. IV delivered buffer therapy is not a practical long-term solution. The inventors have demonstrated that topically delivered buffer therapy has proven to be a viable solution for both short term and long-term use. However, previously described inventions have not accounted for potential challenges of choric therapy; most notably cation overload and electrolyte imbalance.

The inventions described herein provide for a method of safe and effective topical delivery of buffering agents. In addition to topically delivering buffers systemically, this invention includes formulations and methods to balance electrolytes and/or deliver buffers without counterions. These elements can be combined in a single use formulation or alternatively in separately applied formulations.

In one aspect, a formulation for transdermal delivery through the skin of a subject comprising a counter ion free buffering compound is provided where the buffering compound is present in an effective amount to ameliorate or counteract an electrolyte or ion imbalance in a subject. Examples of suitable buffering agents provided herein include Lysine (free base), TRIS, and IEPA.

An exemplary embodiment of the formulation comprises TRIS base in an amount of about 20.0% w/w to about 40.0% w/w; and optionally one or more of the following menthol in an amount of about 0.20% w/w to about 1.0% w/w; cetyl alcohol in an amount of about 1.0% w/w to about 5.0% w/w; almond oil in an amount of about 1.0% w/w to about 5.0% w/w; lipmax® in an amount of about 10.0% w/w to about 25.0% w/w; benzyl alcohol in an amount of about 0.5% w/w to about 5.0% w/w; poloxamer 407 (Pluronic®) in an amount of about 3.0% w/w to about 20.0% w/w; glycerine in an amount of about 0.2% w/w to about 1.0% w/w; propylene glycol in an amount of about 1.0% w/w to about 5.0% w/w; NaOH (50% solution) in an amount of about 0.1% w/w to about 5.0% w/w; ethanol in an amount of about 0.5% w/w to about 3.0% w/w; and water in an amount to complete.

In another aspect, a formulation for transdermal delivery through the skin of a subject comprising one or more buffering compound is provided in which at least one buffering compound has a counterion that is a cation and the one or more buffering compounds are present in a ratio and amount effective to ameliorate or counteract an electrolyte or ion imbalance in the subject. Suitable buffering agents include, for example, potassium bicarbonate, sodium bicarbonate, calcium carbonate, magnesium carbonate, and potassium carbonate.

An exemplary embodiment comprises calcium carbonate in an amount of about 5.0% w/w to about 30.0% w/w and sodium bicarbonate in an amount of about 5.0% w/w to about 30.0% w/w, and optionally one or more of the following menthol in an amount of about 0.20% w/w to about 1.0% w/w; cetyl alcohol in an amount of about 1.0% w/w to about 5.0% w/w; almond oil in an amount of about 1.0% w/w to about 5.0% w/w; lipmax® in an amount of about 10.0% w/w to about 25.0% w/w; benzyl alcohol in an amount of about 0.5% w/w to about 5.0% w/w; poloxamer 407 (Pluronic®) in an amount of about 3.0% w/w to about 20.0% w/w; glycerine in an amount of about 0.2% w/w to about 1.0% w/w; propylene glycol in an amount of about 1.0% w/w to about 5.0% w/w; NaOH (50% solution) in an amount of about 0.1% w/w to about 5.0% w/w; ethanol in an amount of about 0.5% w/w to about 3.0% w/w; and water in an amount to complete.

In another aspect, a method of treating an electrolyte imbalance in a patient in need thereof is provided comprising administering a formulation provided herein.

In another aspect, a method of treating a cation overload or imbalance in a patient in need thereof comprising administering a formulation provided herein.

In another aspect, a method of treating a cation overload or imbalance in a patient caused by another transdermally administered formulation is provided comprising administering a formulation provided herein.

In another aspect, a method of treating an oncology patient having a tumor lysis syndrome (TLS) is provided comprising administering a formulation provided herein.

Other formulations and conditions and disorders can be treated by the formulations are described in greater detail below.

### DETAILED DESCRIPTION

The practices described herein employ, unless otherwise indicated, conventional techniques of tissue culture, immunology, molecular biology, microbiology, cell biology and recombinant DNA, which are within the skill of the art. See, e.g., Harlow and Lane eds. (1999) Antibodies, A Laboratory Manual and Herzenberg et al. eds (1996) Weir's Handbook of Experimental Immunology.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a pharmaceutically acceptable carrier” includes a plurality of pharmaceutically acceptable carriers, including mixtures thereof. On the other hand “one” designates the singular.

As used herein, the term “comprising” is intended to mean that the compositions and methods include the listed elements, but do not exclude other unlisted elements. “Consisting essentially of” when used to define compositions and methods, excludes other elements that alters the basic nature of the composition and/or method, but does not exclude other unlisted elements. Thus, a composition consisting essentially of the elements as defined herein would not exclude trace amounts of elements, such as contaminants from any isolation and purification methods or pharmaceutically acceptable carriers, such as phosphate buffered saline, preservatives, and the like, but would exclude additional unspecified amino acids. “Consisting of” excludes more than trace elements of other ingredients and substantial method steps for administering the compositions described herein. Embodiments defined by each of these transition terms are within the scope of this disclosure and the inventions embodied therein.

Many known and useful compounds and the like can be found in Remington's Pharmaceutical Sciences (13^{th} Ed), Mack Publishing Company, Easton, Pa.—a standard reference for various types of administration. As used herein, the term “formulation(s)” means a combination of at least one active ingredient with one or more other ingredient, also commonly referred to as excipients, which may be independently active or inactive. The term “formulation”, may or may not refer to a pharmaceutically acceptable composition for administration to humans or animals, and may include compositions that are useful intermediates for storage or research purposes.

As the patients and subjects of the invention method are, in addition to humans, veterinary subjects, formulations suitable for these subjects are also appropriate. Such subjects include livestock and pets as well as sports animals such as horses, greyhounds, and the like.

In an embodiment, a “pharmaceutical composition” is intended to include, without limitation, the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

In an embodiment, “an effective amount” refers, without limitation, to the amount of the defined component sufficient to achieve the desired chemical composition or the desired biological and/or therapeutic result. In an embodiment, that result can be the desired pH or chemical or biological characteristic, e.g., stability of the formulation. In other embodiments, the desired result is the alleviation or amelioration of the signs, symptoms, or causes of a disease, or any other desired alteration of a biological system. When the desired result is a therapeutic response, the effective amount will, without limitation, vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art. A desired effected may, without necessarily being therapeutic, also be a cosmetic effect, in particular for treatment for disorders of the skin described herein.

In an embodiment, a “subject” of diagnosis or treatment is, without limitation, a prokaryotic or a eukaryotic cell, a tissue culture, a tissue or an animal, e.g. a mammal, including a human. Non-human animals subject to diagnosis or treatment include, for example, without limitation, a simian, a murine, a canine, a leporid, such as a rabbit, livestock, sport animals, and pets.

In an embodiment, as used herein, the terms “treating,” “treatment” and the like are used herein, without limitation, to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

There are difficult challenges to develop formulations for topical delivery for counteracting or treating unwanted cation overload and electrolyte imbalances that apparently have not been reported or studied previously. One challenge relates to the topical delivery of effective amounts of therapeutic agents or compounds of the formulations (e.g. buffers), which has not been successfully reported.

Additionally, the use of particular formulations can disrupt the balance of electrolytes and cations, including those such as the Na/K ratio. For example, the administration of formulations containing calcium carbonate can reduce the amount of sodium or other ions which can decrease the potential for reaching a hyponatremic state. Also, the use of calcium carbonate can also increase the serum levels of calcium which can reduce the amount of calcium leeched from the body by high sodium concentrations.

Electrolytes must exist in the body within a narrow concentration range in order to effectively serve a variety of important and/or critical functions (see Table 1). The normal range is measured per liter of blood. Electrolyte imbalance refers to a value higher or lower than the normal range and can cause a variety of symptoms.

TABLE 1
*Values may vary from laboratory to laboratory.

| Electrolyte | Functions in the body | Normal adult range* | |
|---|---|---|---|
| Calcium | Necessary for muscle contraction, | 4.5-5.5 | mEq/L |
| | nerve function, blood clotting, cell | | |
| | division, healthy bones and teeth | | |
| Chloride | Maintains fluid balance in the body | 97-107 | mEq/L |
| Potassium | Regulates heart contraction, | 3.5-5.3 | mEq/L |
| | helps maintain fluid balance | | |
| Magnesium | Necessary for muscle contraction, | 1.5-2.5 | mEq/L |
| | nerve function, heart rhythm, bone | | |
| | strength, generating energy and | | |
| | building protein | | |
| Sodium | Maintains fluid balance and | 136-145 | mEq/L |
| | necessary for muscle contraction | | |
| | and nerve function | | |

Electrolyte imbalance is commonly caused by loss of body fluids through prolonged vomiting, diarrhea, sweating, or high fever. These conditions and disorders may be side effects of chemotherapy or other oncology treatments such as electrolyte imbalances caused by buffering therapy (e.g. pH modulating formulations). Other potential toxic side effects of chronic sodium bicarbonate ingestion include, for example, hypernatremia, hypokalemia, hypochloremia, hypertension, gastric rupture, intravascular volume depletion, headache, loss of appetite, mood or mental changes, muscle pain or twitching, nausea or vomiting, nervousness or restlessness, slow breathing, swelling of feet or lower legs, unpleasant taste, unusual tiredness or weakness. Increase blood serum sodium levels can leech also calcium from body including bones which can cause osteoporosis.

Some patients with tumor lysis syndrome may have initial symptoms including: nausea and vomiting, joint discomfort, shortness of breath, irregular heartbeat, lethargy, and cloudy urine. Other patients feel no symptoms of tumor lysis syndrome in its early stages but have abnormal laboratory results. Laboratory results indicative of tumor lysis syndrome show high potassium, uric acid and phosphorous levels and low calcium levels in the blood.

In oncology patients in particular, the electrolyte balance is further complicated by tumor lysis syndrome. When cancer cells are killed by therapy, they may spill their inner (intracellular) contents, which accumulate in the body faster than can be eliminated. These excess intracellular contents cause the metabolic and electrolyte disturbances that result in tumor lysis syndrome (TLS). Tumor lysis syndrome can result in life-threatening complications if not managed appropriately. If tumor lysis syndrome is untreated, its progression may cause acute kidney failure, cardiac arrhythmias, seizures, loss of muscle control or death.

Patients at a high risk of developing tumor lysis syndrome typically have acute leukemia or lymphoma that is very responsive to chemotherapy. Patients with pre-existing kidney dysfunction are also at an increased risk of developing tumor lysis syndrome. Patients who are considered to be at risk of developing tumor lysis syndrome are typically treated with preventive measures prior to and during their treatment for cancer. Preventive measures typically include intravenous hydration, medications including allopurinol or Elitek® (rasburicase), and alkalinization of the urine with sodium bicarbonate. Once tumor lysis syndrome has actually developed, a patient needs to also be treated for the specific medical abnormality that is present, which typically includes one of the following: high uric acid (hyperuricemia), high potassium (hyperkalemia), high phosphate (hyperphosphatemia), and low calcium (hypocalcemia). Applicant's recently disclosed approaches to buffer therapy utilize sodium bicarbonate which is quite effective for the treatment of cancer and related conditions; however, the potential for hypernatremia is a significant side effect in some patients.

The formulations and methods of treatment provided herein overcome these challenges and are useful for counteracting or treating unwanted cation overload and electrolyte imbalances and associated conditions and disorders. The formulations and methods of treatment provided herein are primarily for topical and transdermal delivery through the skin of a subject; however other types of formulations and routes of delivery are envisioned.

While calcium carbonate is used to treat high phosphate, tumor lysis syndrome often results in low calcium and high phosphate. An aspect of the invention is to provide formulations that incorporate calcium carbonate in order to counteract these deficiencies while also providing the benefits of buffer therapy.

Another condition that can be treated by formulations and methods described herein relates to high or abnormal levels of uric acid. High uric acid can be a result of decreased kidney function and is exacerbated in low pH, high acidity, environments. An aspect of the invention is to provide buffering therapy formulations to counteract, inhibit, or prevent the side effects of kidney function caused by high or undesirable amounts of uric acid.

Another condition that can be treated by formulations and methods described herein relates to high or abnormal levels of potassium. High potassium is caused as cells are destroyed and potassium moves into the bloodstream. Another aspect of the invention is to provide formulations, comprising, for example, sodium bicarbonate, for administration to counteract acidosis and to promote movement of potassium from the extracellular space back into the cells

The formulations and methods of use provided herein take these complexities of electrolyte balance into account. One approach utilized herein in making formulations that avoid electrolyte imbalance and cation overload is to use non-metal buffers or buffers without counterions. Suitable buffering agents for these embodiments include Lysine (free base), TRIS, and IEPA.

Another approach to make electrolyte balancing formulations is to avoid electrolyte imbalances by incorporating different buffers in different amount or ratios. Non-limiting examples of buffering agents that can be used together in different amounts or ratios include potassium bicarbonate, sodium bicarbonate, calcium carbonate, magnesium carbonate, and potassium carbonate. Mixtures of particular buffering agents including 2, 3, 4, 5, or more buffering agents are used depending on the formulation. Further, the relative amounts or ratio of each buffering agent may vary, for example, where the relative amounts are from 1:1.10 w/w; 1:1.15 w/w; 1:1.20 w/w; 1:1.25 w/w; 1:1.30 w/w; 1:1.35 w/w; 1:1.40 w/w; 1:1.45 w/w; 1:1.50 w/w; 1:1.55 w/w; 1:1.60 w/w; 1:1.65 w/w; 1:1.70 w/w; 1:1.75 w/w; 1:1.80 w/w; 1:1.85 w/w; 1:1.90 w/w; 1:1.95 w/w; 1:2 w/w; 1:2.5 w/w; 1:3 w/w; 1:3.5 w/w; 1:4 w/w, 1:4.5 w/w; 1:5 w/w, 1:5.5 w/w; 1:6 w/w; 1:6.5 w/w; 1:7 w/w; 1:8 w/w; 1:9 w/w; or 1:10 w/w. These ratios of buffering agents are applicable when two buffering agents are present, or more than two and the ratios are applicable between any two buffering agents.

A suitable non-limiting electrolyte balancing topical formulation may, for example, comprise potassium bicarbonate in an amount of between about 30% to about 40%, magnesium carbonate in an amount of between about 35% to about 50%, and sodium bicarbonate in an amount of between about 15% and up to about 30%, relative to each of the others w/w.

Another suitable electrolyte balancing topical formulation may comprise calcium carbonate in an amount of about 85% and potassium carbonate in an amount of about 15%, relative to each other w/w. In other embodiments, the relative amounts of calcium carbonate and potassium carbonate are 90% and 10%, 80% and 20%, 75% to 25%, 70% to 30%, 65% to 35%, 60% to 40%, 55% to 45%, and 50% to 50%.

Another suitable electrolyte balancing topical formulation may comprise sodium bicarbonate in an amount of about 50% and potassium bicarbonate in an amount of about 50%, relative w/w. Another suitable electrolyte balancing topical formulation may comprise sodium bicarbonate 40-60%, calcium carbonate 40-60%, relative w/w.

In some embodiments, the formulation comprises up to about 70.0% w/w of a mixture consisting of between 30% to about 40% potassium bicarbonate, about 50% magnesium carbonate, and between about 15% to about 30% sodium bicarbonate, w/w and:
Menthol in an amount of 0.0% w/w up to about 5.0% w/w;
Cetyl alcohol in an amount of 0.0% w/w up to about 5.0% w/w;
Almond oil in an amount of 0.0% w/w up to about 5.0% w/w;
Lipmax® in an amount of 0.0% w/w up to about 30.0% w/w;
Benzyl alcohol in an amount of 0.0% w/w up to about 5.0% w/w;
Poloxamer 407 (Pluronic®) in an amount of 0.0% w/w up to about 20.0% w/w;
Glycerine in an amount of 0.0% w/w up to about 2.0% w/w;
Propylene glycol in an amount of 0.0% w/w up to about 10.0% w/w;
NaOH (50% solution) in an amount of 0.0% w/w up to about 5.0% w/w;
Ethanol in an amount of 0.0% w/w up to about 5.0% w/w;
Water in an amount to complete.

In some embodiments, the formulation comprises up to about 70.0% w/w of a mixture consisting of about 85% calcium carbonate and 15% potassium carbonate, w/w, and
Menthol in an amount of 0.0% w/w up to about 5.0% w/w;
Cetyl alcohol in an amount of 0.0% w/w up to about 5.0% w/w;
Almond oil in an amount of 0.0% w/w up to about 5.0% w/w;
Lipmax® in an amount of 0.0% w/w up to about 30.0% w/w;
Benzyl alcohol in an amount of 0.0% w/w up to about 5.0% w/w;
Poloxamer 407 (Pluronic®) in an amount of 0.0% w/w up to about 20.0% w/w;
Glycerine in an amount of 0.0% w/w up to about 2.0% w/w;
Propylene glycol in an amount of 0.0% w/w up to about 10.0% w/w;
NaOH (50% solution) in an amount of 0.0% w/w up to about 5.0% w/w;
Ethanol in an amount of 0.0% w/w up to about 5.0% w/w;
Water in an amount to complete.

In some embodiments, the formulation comprises up to about 70.0% w/w of a mixture consisting of about 50% sodium bicarbonate and 50% potassium bicarbonate, w/w, and
Menthol in an amount of 0.0% w/w up to about 5.0% w/w;
Cetyl alcohol in an amount of 0.0% w/w up to about 5.0% w/w;
Almond oil in an amount of 0.0% w/w up to about 5.0% w/w;
Lipmax® in an amount of 0.0% w/w up to about 30.0% w/w;
Benzyl alcohol in an amount of 0.0% w/w up to about 5.0% w/w;
Poloxamer 407 (Pluronic®) in an amount of 0.0% w/w up to about 20.0% w/w;
Glycerine in an amount of 0.0% w/w up to about 2.0% w/w;
Propylene glycol in an amount of 0.0% w/w up to about 10.0% w/w;
NaOH (50% solution) in an amount of 0.0% w/w up to about 5.0% w/w;
Ethanol in an amount of 0.0% w/w up to about 5.0% w/w;
Water in an amount to complete.

In some embodiments, the formulation comprises up to about 70.0% w/w of a mixture consisting of between 40% to about 60% sodium bicarbonate and between about 40% to about 60% calcium carbonate, and
Menthol in an amount of 0.0% w/w up to about 5.0% w/w;
Cetyl alcohol in an amount of 0.0% w/w up to about 5.0% w/w;
Almond oil in an amount of 0.0% w/w up to about 5.0% w/w;
Lipmax® in an amount of 0.0% w/w up to about 30.0% w/w;
Benzyl alcohol in an amount of 0.0% w/w up to about 5.0% w/w;
Poloxamer 407 (Pluronic®) in an amount of 0.0% w/w up to about 20.0% w/w;
Glycerine in an amount of 0.0% w/w up to about 2.0% w/w;
Propylene glycol in an amount of 0.0% w/w up to about 10.0% w/w;
NaOH (50% solution) in an amount of 0.0% w/w up to about 5.0% w/w;
Ethanol in an amount of 0.0% w/w up to about 5.0% w/w;
Water in an amount to complete.

In some embodiments, the formulation comprises:
Calcium carbonate in an amount of about 0.0% w/w to about 50.0% w/w;
Menthol in an amount of 0.0% w/w up to about 5.0% w/w;
Cetyl alcohol in an amount of 0.0% w/w up to about 5.0% w/w;
Almond oil in an amount of 0.0% w/w up to about 5.0% w/w;
Lipmax® in an amount of 0.0% w/w up to about 30.0% w/w;
Benzyl alcohol in an amount of 0.0% w/w up to about 5.0% w/w;
Poloxamer 407 (Pluronic®) in an amount of 0.0% w/w up to about 20.0% w/w;
Glycerine in an amount of 0.0% w/w up to about 2.0% w/w;
Propylene glycol in an amount of 0.0% w/w up to about 10.0% w/w;
NaOH (50% solution) in an amount of 0.0% w/w up to about 5.0% w/w;
Ethanol in an amount of 0.0% w/w up to about 5.0% w/w; and
Water in an amount to complete.

In some embodiments, the formulation comprises:
Calcium carbonate in an amount of about 10.0% w/w to about 40.0% w/w; and optionally one or more of the following:
Menthol in an amount of about 0.10% w/w to about 5.0% w/w;
Cetyl alcohol in an amount of about 0.5% w/w to about 5.0% w/w;
Almond oil in an amount of about 0.5% w/w to about 5.0% w/w;
Lipmax® in an amount of about 5.0% w/w to about 30.0% w/w;
Benzyl alcohol in an amount of about 0.1% w/w to about 5.0% w/w;
Poloxamer 407 (Pluronic®) in an amount of about 3.0% w/w to about 20.0% w/w;
Glycerine in an amount of about 0.1% w/w to about 2.0% w/w;
Propylene glycol in an amount of about 0.5% w/w to about 10.0% w/w;
NaOH (50% solution) in an amount of about 0.1% w/w to about 5.0% w/w;
Ethanol in an amount of about 0.3% w/w to about 5.0% w/w; and
Water in an amount to complete.

In some embodiments, the formulation comprises:
Calcium carbonate in an amount of about 10.0% w/w to about 40.0% w/w; and optionally one or more of the following:
Menthol in an amount of about 0.20% w/w to about 1.0% w/w;
Cetyl alcohol in an amount of about 1.0% w/w to about 5.0% w/w;
Almond oil in an amount of about 1.0% w/w to about 5.0% w/w;
Lipmax® in an amount of about 10.0% w/w to about 25.0% w/w;
Benzyl alcohol in an amount of about 0.5% w/w to about 5.0% w/w;
Poloxamer 407 (Pluronic®) in an amount of about 3.0% w/w to about 20.0% w/w;
Glycerine in an amount of about 0.2% w/w to about 1.0% w/w;
Propylene glycol in an amount of about 1.0% w/w to about 5.0% w/w;
NaOH (50% solution) in an amount of about 0.1% w/w to about 5.0% w/w;
Ethanol in an amount of about 0.5% w/w to about 3.0% w/w; and
Water in an amount to fill/complete.

In a particular embodiment, the formulation comprises:
Calcium carbonate in an amount of about 25.0% w/w;
and optionally one or more of the following:
Menthol in an amount of about 0.50% w/w;
Cetyl alcohol in an amount of about 2.0% w/w;
Almond oil in an amount of about 3.0% w/w;
Lipmax® in an amount of about 18.0% w/w;
Benzyl alcohol in an amount of about 1.0% w/w;
where the above components comprise a first phase (A) of about 24.5% w/w; and
water in an amount of about 29.0% w/w;
Poloxamer 407 (Pluronic®) in an amount of about 15.0% w/w;
Glycerine in an amount of about 0.5% w/w;
Propylene glycol in an amount of about 4.0% w/w;
NaOH (50% solution) in an amount of about 0.5% w/w; and
Ethanol in an amount of about 1.5% w/w.

The above formula can be represented in Table 2 below.

TABLE 2

| Ingredient | wt % |
|---|---|
| Menthol | 0.50% |
| Cetyl Alcohol | 2.00% |
| Almond Oil | 3.00% |
| Lipmax | 18.00% |
| Benzyl Alcohol | 1.00% |
| Total Phase A | 24.50% |
| Water | 29.00% |
| Pluronic Gel | 15.00% |
| Glycerine | 0.50% |
| Propylene Glycol | 4.00% |
| NaOH 50% sol. | 0.50% |
| EtOH | 1.50% |
| CaCarb | 25.00% |
| Total | 100.00% |

Another embodiment of a formulation according to the invention is represented in Table 3 below:

TABLE 3

| Ingredient | wt % |
|---|---|
| Menthol | 0.50% |
| Cetyl Alcohol | 2.00% |
| Almond Oil | 3.00% |
| Lipmax | 18.00% |
| Benzyl Alcohol | 1.00% |
| Total Phase A | 24.50% |
| Water | 22.00% |
| Pluronic Gel | 15.00% |
| Glycerine | 0.50% |
| Propylene Glycol | 4.00% |
| NaOH 50% sol. | 0.50% |
| EtOH | 1.50% |
| CaCarb | 32.00% |
| Total | 100.00% |

Another embodiment of a formulation according to the invention is represented in Table 4 below:

TABLE 4

| Ingredient | wt % |
|---|---|
| Menthol | 0.50% |
| Cetyl Alcohol | 2.00% |
| Almond Oil | 3.00% |
| Lipmax | 18.00% |
| Benzyl Alcohol | 1.00% |
| Total Phase A | 24.50% |
| Water | 22.00% |
| Pluronic Gel | 15.00% |
| Glycerine | 0.50% |
| Propylene Glycol | 4.00% |
| NaOH 50% sol. | 0.50% |
| EtOH | 1.50% |
| CaCarb | 32.00% |
| Total | 100.00% |

In some embodiments, the formulation comprises:
Calcium carbonate in an amount of about 0.0% w/w to about 30.0% w/w;
Sodium bicarbonate in an amount of about 0.0% w/w to about 30.0% w/w;
Menthol in an amount of 0.0% w/w up to about 5.0% w/w;
Cetyl alcohol in an amount of 0.0% w/w up to about 5.0% w/w;
Almond oil in an amount of 0.0% w/w up to about 5.0% w/w;
Lipmax® in an amount of 0.0% w/w up to about 30.0% w/w;
Benzyl alcohol in an amount of 0.0% w/w up to about 5.0% w/w;
Poloxamer 407 (Pluronic®) in an amount of 0.0% w/w up to about 20.0% w/w;
Glycerine in an amount of 0.0% w/w up to about 2.0% w/w;
Propylene glycol in an amount of 0.0% w/w up to about 10.0% w/w;
NaOH (50% solution) in an amount of 0.0% w/w up to about 5.0% w/w;
Ethanol in an amount of 0.0% w/w up to about 5.0% w/w; and
Water in an amount to complete.

In some embodiments, the formulation comprises:
Calcium carbonate in an amount of about 5.0% w/w to about 30.0% w/w;
Sodium bicarbonate in an amount of about 5.0% w/w to about 30.0% w/w;
and optionally one or more of the following:
Menthol in an amount of about 0.10% w/w to about 5.0% w/w;
Cetyl alcohol in an amount of about 0.5% w/w to about 5.0% w/w;
Almond oil in an amount of about 0.5% w/w to about 5.0% w/w;
Lipmax® in an amount of about 5.0% w/w to about 30.0% w/w;
Benzyl alcohol in an amount of about 0.1% w/w to about 5.0% w/w;
Poloxamer 407 (Pluronic®) in an amount of about 3.0% w/w to about 20.0% w/w;
Glycerine in an amount of about 0.1% w/w to about 2.0% w/w;
Propylene glycol in an amount of about 0.5% w/w to about 10.0% w/w;
NaOH (50% solution) in an amount of about 0.1% w/w to about 5.0% w/w;
Ethanol in an amount of about 0.3% w/w to about 5.0% w/w; and
Water in an amount to complete.

In some embodiments, the formulation comprises
Calcium carbonate in an amount of about 5.0% w/w to about 30.0% w/w;
Sodium bicarbonate in an amount of about 5.0% w/w to about 30.0% w/w;
and optionally one or more of the following:
Menthol in an amount of about 0.20% w/w to about 1.0% w/w;
Cetyl alcohol in an amount of about 1.0% w/w to about 5.0% w/w;
Almond oil in an amount of about 1.0% w/w to about 5.0% w/w;
Lipmax® in an amount of about 10.0% w/w to about 30.0% w/w;
Benzyl alcohol in an amount of about 0.5% w/w to about 5.0% w/w;
Poloxamer 407 (Pluronic®) in an amount of about 3.0% w/w to about 20.0% w/w;
Glycerine in an amount of about 0.2% w/w to about 1.0% w/w;
Propylene glycol in an amount of about 1.0% w/w to about 5.0% w/w;
NaOH (50% solution) in an amount of about 0.1% w/w to about 5.0% w/w;
Ethanol in an amount of about 0.5% w/w to about 3.0% w/w; and
Water in an amount to fill/complete.

In a particular embodiment represented in Table 5 below, the formulation comprises:
Sodium Bicarbonate (milled) in an amount of about 12.5% w/w;
Calcium carbonate in an amount of about 12.5% w/w;
Menthol in an amount of about 0.50% w/w;
Cetyl alcohol in an amount of about 2.0% w/w;
Almond oil in an amount of about 3.0% w/w;
Lipmax® in an amount of about 18.0% w/w;
Benzyl alcohol in an amount of about 1.0% w/w;
where the above components comprise a first phase (A) of about 24.5% w/w; and
water in an amount of about 29.0% w/w;
Poloxamer 407 (Pluronic®) in an amount of about 15.0% w/w;
Glycerine in an amount of about 0.5% w/w;
Propylene glycol in an amount of about 4.0% w/w;
NaOH (50% solution) in an amount of about 0.5% w/w; and
Ethanol in an amount of about 1.5% w/w.

TABLE 5

| Chemicals | wt % |
|---|---|
| Menthol | 0.50% |
| Cetyl Alcohol | 2.00% |
| Almond Oil | 3.00% |
| Lipmax | 18.00% |
| Benzyl Alcohol | 1.00% |
| Total Phase A | 24.50% |
| Water | 29.00% |
| Pluronic Gel | 15.00% |
| Glycerine | 0.50% |
| Propylene Glycol | 4.00% |
| NaOH 50% sol. | 0.50% |
| EtOH | 1.50% |
| Sodium Bicarb. | 12.50% |
| (Milled) | |
| CaCarb | 12.50% |
| Total | 100% |

Another embodiment of a formulation according to the invention is represented in Table 6 below:

TABLE 6

| Chemicals | wt % |
|---|---|
| Menthol | 0.50% |
| Cetyl Alcohol | 2.00% |
| Almond Oil | 3.00% |
| Lipmax | 18.00% |
| Benzyl Alcohol | 1.00% |
| Total Phase A | 24.50% |
| Water | 22.00% |
| Pluronic Gel | 15.00% |
| Glycerine | 0.50% |
| Propylene Glycol | 4.00% |
| NaOH 50% sol. | 0.50% |
| EtOH | 1.50% |
| Sodium Bicarb. | 16.00% |
| (Milled) | |
| CaCarb | 16.00% |
| Total | 100% |

Another embodiment of a formulation according to the invention is represented in Table 7 below:

TABLE 7

| Chemicals | wt % |
|---|---|
| Menthol | 0.50% |
| Cetyl Alcohol | 1.00% |
| Almond Oil | 3.00% |
| Lipmax | 18.00% |
| Benzyl Alcohol | 1.00% |
| Total Phase A | 23.50% |
| Water | 35.00% |
| Pluronic Gel | 10.00% |
| Glycerine | 0.50% |
| Propylene Glycol | 4.00% |
| NaOH 50% sol. | 0.50% |
| EtOH | 1.50% |
| Sodium Bicarb. | 12.50% |
| (Milled) | |
| CaCarb | 12.50% |
| Total | 100% |

Another embodiment of a formulation according to the invention is represented in Table 8 below:

TABLE 8

| Chemicals | wt % |
|---|---|
| Menthol | 0.50% |
| Cetyl Alcohol | 1.00% |
| Almond Oil | 3.00% |
| Lipmax | 18.00% |
| Benzyl Alcohol | 1.00% |
| Water | 28.00% |
| Pluronic Gel | 10.00% |
| Glyerine | 0.50% |
| Propylene Glycol | 4.00% |
| NaOH 50% sol. | 0.50% |
| EtOH | 1.50% |
| Sodium Bicarb. | 16.00% |
| (Milled) | |
| CaCarb | 16.00% |
| Total | 100% |

In another aspect, certain embodiments of the formulations use buffers which do not have counter ions and thus have reduced or eliminated the risk of hypernatremia. Tris-base buffers have other potentially beneficial characteristics including a demonstrated antitumor effect in vivo. Accordingly, certain embodiments of the formulation incorporate a Tris-base in an amount of up to about 60.0% w/w; up to about 50.0% w/w; up to about 45.0% w/w; up to about 40.0% w/w; up to about 35.0% w/w; up to about 30.0% w/w; up to about 25.0% w/w; up to about 20.0% w/w; up to about 17.0% w/w; up to about 15.0% w/w; up to about 10.0% w/w; or up to about 5.0% w/w. Other amounts are possible. The formulation typically comprises one or more compound.

In some embodiments, the formulation comprises Tris-base in an amount of up to 50.0% w/w; and one or more of the following:
Menthol in an amount of 0.0% w/w up to about 5.0% w/w;
Cetyl alcohol in an amount of 0.0% w/w up to about 5.0% w/w;
Almond oil in an amount of 0.0% w/w up to about 5.0% w/w;
Lipmax® in an amount of 0.0% w/w up to about 30.0% w/w;
Benzyl alcohol in an amount of 0.0% w/w up to about 5.0% w/w;
Poloxamer 407 (Pluronic®) in an amount of 0.0% w/w up to about 20.0% w/w;
Glycerine in an amount of 0.0% w/w up to about 2.0% w/w;
Propylene glycol in an amount of 0.0% w/w up to about 10.0% w/w;
NaOH (50% solution) in an amount of 0.0% w/w up to about 5.0% w/w;
Ethanol in an amount of 0.0% w/w up to about 5.0% w/w; and
Water in an amount to complete.

In some embodiments, the formulation comprises:
Tris-base in an amount of about 10.0% w/w to about 50.0% w/w;
Menthol in an amount of about 0.10% w/w to about 5.0% w/w;
Cetyl alcohol in an amount of about 0.5% w/w to about 5.0% w/w;
Almond oil in an amount of about 0.5% w/w to about 5.0% w/w;
Lipmax® in an amount of about 5.0% w/w to about 30.0% w/w;
Benzyl alcohol in an amount of about 0.1% w/w to about 5.0% w/w;
Poloxamer 407 (Pluronic®) in an amount of about 3.0% w/w to about 20.0% w/w;
Glycerine in an amount of about 0.1% w/w to about 2.0% w/w;
Propylene glycol in an amount of about 0.5% w/w to about 10.0% w/w;
NaOH (50% solution) in an amount of about 0.1% w/w to about 5.0% w/w;
Ethanol in an amount of about 0.3% w/w to about 5.0% w/w; and
Water in an amount to complete.

In some embodiments, the formulation comprises TRIS-base in an amount of about 20.0% w/w to about 40.0% w/w; and optionally one or more of the following:
Menthol in an amount of about 0.20% w/w to about 1.0% w/w;
Cetyl alcohol in an amount of about 1.0% w/w to about 5.0% w/w;
Almond oil in an amount of about 1.0% w/w to about 5.0% w/w;
Lipmax® in an amount of about 10.0% w/w to about 25.0% w/w;
Benzyl alcohol in an amount of about 0.5% w/w to about 5.0% w/w;
Poloxamer 407 (Pluronic®) in an amount of about 3.0% w/w to about 20.0% w/w;
Glycerine in an amount of about 0.2% w/w to about 1.0% w/w;
Propylene glycol in an amount of about 1.0% w/w to about 5.0% w/w;
NaOH (50% solution) in an amount of about 0.1% w/w to about 5.0% w/w;
Ethanol in an amount of about 0.5% w/w to about 3.0% w/w; and
Water in an amount to complete.

In a particular embodiment, the formulation comprises the components in the amounts listed in Table 9.

TABLE 9

| Ingredient | Weight % |
|---|---|
| Menthol | 0.50% |
| Cetyl Alcohol | 2.00% |
| Almond Oil | 3.00% |
| Lipmax ® | 16.00% |
| Benzyl Alcohol | 1.00% |
| Water | 23.50% |
| 30% Pluronic Gel | 15.00% |
| Glycerine | 0.50% |
| Propylene Glycol | 4.00% |
| NaOH 50% Soln. | 0.50% |
| Ethanol | 1.50% |
| Tris-Base | 32.50% |
| Total | 100.00% |

In another aspect, certain embodiments of the formulations use free base lysine buffers. Free base lysine buffers have other potentially beneficial characteristics, including the ability to inhibit metastasis in prostate cancer cells due to alkalization of the extracellular tumor microenvironment. Accordingly, certain embodiments of the formulation incorporate a free base lysine in an amount of up to about 60.0% w/w; up to about 50.0% w/w; up to about 45.0% w/w; up to about 40.0% w/w; up to about 35.0% w/w; up to about 30.0% w/w; up to about 25.0% w/w; up to about 20.0% w/w; up to about 17.0% w/w; up to about 15.0% w/w; up to about 10.0% w/w; or up to about 5.0% w/w. Other amounts are possible. The formulation typically comprises one or more compound.

In some embodiments, the formulation comprises:
Free base lysine in an amount of up to 50.0% w/w;
Menthol in an amount of 0.0% w/w up to about 5.0% w/w;
Cetyl alcohol in an amount of 0.0% w/w up to about 5.0% w/w;
Almond oil in an amount of 0.0% w/w up to about 5.0% w/w;
Lipmax® in an amount of 0.0% w/w up to about 30.0% w/w;
Benzyl alcohol in an amount of 0.0% w/w up to about 5.0% w/w;
Poloxamer 407 (Pluronic®) in an amount of 0.0% w/w up to about 20.0% w/w;
Glycerine in an amount of 0.0% w/w up to about 2.0% w/w;
Propylene glycol in an amount of 0.0% w/w up to about 10.0% w/w;
NaOH (50% solution) in an amount of 0.0% w/w up to about 5.0% w/w;
Ethanol in an amount of 0.0% w/w up to about 5.0% w/w; and
Water in an amount to complete.

In some embodiments, the formulation comprises Lysine (free base) in an amount of about 10.0% w/w to about 50.0% w/w; and optionally one or more of the following:
Menthol in an amount of about 0.10% w/w to about 5.0% w/w;
Cetyl alcohol in an amount of about 0.5% w/w to about 5.0% w/w;
Almond oil in an amount of about 0.5% w/w to about 5.0% w/w;
Lipmax® in an amount of about 5.0% w/w to about 30.0% w/w;
Benzyl alcohol in an amount of about 0.1% w/w to about 5.0% w/w;
Poloxamer 407 (Pluronic®) in an amount of about 3.0% w/w to about 20.0% w/w;
Glycerine in an amount of about 0.1% w/w to about 2.0% w/w;
Propylene glycol in an amount of about 0.5% w/w to about 10.0% w/w;
NaOH (50% solution) in an amount of about 0.1% w/w to about 5.0% w/w;
Ethanol in an amount of about 0.3% w/w to about 5.0% w/w; and
Water in an amount to complete.

In some embodiments, the formulation comprises Lysine (free base) in an amount of about 20.0% w/w to about 40.0% w/w; and optionally one or more of the following:
Menthol in an amount of about 0.20% w/w to about 1.0% w/w;
Cetyl alcohol in an amount of about 1.0% w/w to about 5.0% w/w;
Almond oil in an amount of about 1.0% w/w to about 5.0% w/w;
Lipmax® in an amount of about 10.0% w/w to about 25.0% w/w;
Benzyl alcohol in an amount of about 0.5% w/w to about 5.0% w/w;
Poloxamer 407 (Pluronic®) in an amount of about 3.0% w/w to about 20.0% w/w;
Glycerine in an amount of about 0.2% w/w to about 1.0% w/w;
Propylene glycol in an amount of about 1.0% w/w to about 5.0% w/w;
NaOH (50% solution) in an amount of about 0.1% w/w to about 5.0% w/w;
Ethanol in an amount of about 0.5% w/w to about 3.0% w/w; and
Water in an amount to complete.

In another aspect, certain embodiments of the formulations use 2-imidazole-1-yl-3-ethoxycarbonylpropionic acid (IEPA). 2-imidazole-1-yl-3-ethoxycarbonylpropionic acid (IEPA) buffers do not have counter ions and thus do not cause risk of hypernatremia. IEPA buffers have other potentially beneficial characteristics, including the ability to reduce tumor acidity in prostate cancer cells in a murine model and reduce metastasis. Accordingly, certain embodiments of the formulation incorporate IEPA in an amount of up to about 60.0% w/w; up to about 50.0% w/w; up to about 45.0% w/w; up to about 40.0% w/w; up to about 35.0% w/w; up to about 30.0% w/w; up to about 25.0% w/w; up to about 20.0% w/w; up to about 17.0% w/w; up to about 15.0% w/w; up to about 10.0% w/w; or up to about 5.0% w/w. Other amounts are possible. The formulation typically comprises one or more compound.

In some embodiments, the formulation comprises:
IEPA in an amount of up to 50.0% w/w;
Menthol in an amount of 0.0% w/w up to about 5.0% w/w;
Cetyl alcohol in an amount of 0.0% w/w up to about 5.0% w/w;
Almond oil in an amount of 0.0% w/w up to about 5.0% w/w;
Lipmax® in an amount of 0.0% w/w up to about 30.0% w/w;
Benzyl alcohol in an amount of 0.0% w/w up to about 5.0% w/w;
Poloxamer 407 (Pluronic®) in an amount of 0.0% w/w up to about 20.0% w/w;
Glycerine in an amount of 0.0% w/w up to about 2.0% w/w;
Propylene glycol in an amount of 0.0% w/w up to about 10.0% w/w;
NaOH (50% solution) in an amount of 0.0% w/w up to about 5.0% w/w;
Ethanol in an amount of 0.0% w/w up to about 5.0% w/w; and
Water in an amount to complete.

In some embodiments, the formulation comprises:
IEPA in an amount of about 10.0% w/w to about 50.0% w/w;
Menthol in an amount of about 0.10% w/w to about 5.0% w/w;
Cetyl alcohol in an amount of about 0.5% w/w to about 5.0% w/w;
Almond oil in an amount of about 0.5% w/w to about 5.0% w/w;
Lipmax® in an amount of about 5.0% w/w to about 30.0% w/w;
Benzyl alcohol in an amount of about 0.1% w/w to about 5.0% w/w;
Poloxamer 407 (Pluronic®) in an amount of about 3.0% w/w to about 20.0% w/w;
Glycerine in an amount of about 0.1% w/w to about 2.0% w/w;
Propylene glycol in an amount of about 0.5% w/w to about 10.0% w/w;
NaOH (50% solution) in an amount of about 0.1% w/w to about 5.0% w/w;
Ethanol in an amount of about 0.3% w/w to about 5.0% w/w; and
Water in an amount to complete.

In some embodiments, the formulation comprises IEPA in an amount of about 20.0% w/w to about 40.0% w/w; and optionally one or more of the following:
Menthol in an amount of about 0.20% w/w to about 1.0% w/w;
Cetyl alcohol in an amount of about 1.0% w/w to about 5.0% w/w;
Almond oil in an amount of about 1.0% w/w to about 5.0% w/w;
Lipmax® in an amount of about 10.0% w/w to about 25.0% w/w;
Benzyl alcohol in an amount of about 0.5% w/w to about 5.0% w/w;
Poloxamer 407 (Pluronic®) in an amount of about 3.0% w/w to about 20.0% w/w;
Glycerine in an amount of about 0.2% w/w to about 1.0% w/w;
Propylene glycol in an amount of about 1.0% w/w to about 5.0% w/w;
NaOH (50% solution) in an amount of about 0.1% w/w to about 5.0% w/w;
Ethanol in an amount of about 0.5% w/w to about 3.0% w/w; and
Water in an amount to complete.

Certain components or ingredients of formulations provided herein may be supplemented with formulation components described in greater detail in the inventor's related applications mentioned above, including U.S. application Ser. No. 16/132,358 filed Sep. 14, 2018, entitled ‘Methods and Formulations For Transdermal Administration Of Buffering Agents’, International Patent Application No. PCT/US18/51250 filed Sep. 14, 2018, entitled ‘Methods of Administration and Treatment’, and International Patent Application PCT/US18/28017 by Bruce Sand filed Apr. 17, 2018, entitled ‘Parental non-systemic administration of buffering agents for inhibiting metastasis of solid tumors, hyperpigmentation and gout’, all incorporated by reference in their entirety herein.

For topical administration, and in particular transdermal administration, the formulation will comprise penetrants including either or both chemical penetrants (CPEs) and peptide-based cellular penetrating agents (CPPs) that encourage transmission across the dermis and/or across membranes including cell membranes, as would be the case in particular for administration by suppository or intranasal administration, but for transdermal administration as well. Particularly suitable penetrants especially for those that contain at least one agent other than buffer include those that are described in the US2009/0053290, WO2014/209910, and WO2017/127834, incorporated by reference herein. In addition to formulations with penetrants, transdermal delivery can be affected by mechanically disrupting the surface of the skin to encourage penetration, or simply by supplying the formulation applied to the skin under an occlusive patch.

Alternatively, the penetrant portion comprises a completion component as well as one or more electrolytes sufficient to impart viscosity and viscoelasticity, one or more surfactants and an alcohol. The completion component can be a polar liquid, a non-polar liquid or an amphiphilic substance. The penetrant may further comprise a keratinolytic agent effective to reduce thiol linkages, disrupt hydrogen bonding and/or effect keratin lysis and/or a cell penetrating peptide (sometimes referred to as a skin-penetrating peptide) and/or a permeation enhancer.

Lecithin organogel is a combination of lecithin with a gelling component, which is typically amphiphilic. Suitable gelling components also include isopropyl palmitate, ethyl laurate, ethyl myristate and isopropyl myristate. In some embodiments, the formulation comprises a gelling agent in an amount less than 5% w/w of the formulation. Certain hydrocarbons, such as cyclopentane, cyclooctane, trans-decalin, trans-pinane, n-pentane, n-hexane, n-hexadecane may also be used. Thus, an important permeation agent is a lecithin organogel, wherein the combination resulting from lecithin and the organic solvent acts as a permeation agent. In some embodiments, the penetrant portion comprises lecithin organogel, an alcohol, a surfactant, and a polar solvent. In some embodiments, the lecithin organogel is a combination of soy lecithin and isopropyl palmitate. In some embodiments, the penetrant portion comprises lecithin and isopropyl palmitate, undecane, isododecane, isopropyl stearate, or a combination thereof. In some embodiments, the formulation comprises Lipmax™ (sold by Lucas Meyer Cosmetics) in an amount between about 1-20% w/w or an equivalent 50/50 mixture of isopropyl palmitate and lecithin. Lecithin organogels are clear, thermodynamically stable, viscoelastic, and biocompatible jelly-like phases composed of hydrated phospholipids and appropriate organic liquid. An example of a suitable lecithin organogel is lecithin isopropyl palmitate, which is formed when isopropyl palmitate is used to dissolve lecithin. The ratio of lecithin to isopropyl palmitate may be 50:50. Illustrated below in the Examples is a formulation containing soy lecithin in combination with isopropyl palmitate; however, other lecithins could also be used such as egg lecithin or synthetic lecithins. Various esters of long chain fatty acids may also be included. Methods for making such lecithin organogels are well known in the art. In most embodiments, the lecithin organogel is present in the final formulation is less than about 20% w/w. In those compositions used to dissolve fat deposits, to alleviate pain from fat removal or in anhydrous compositions, the concentration of lecithin organogel may be as low as 0.5% w/w, 1% w/w, 5% w/w, 10% w/w or 20% w/w. In some embodiments, the penetrant portion comprises a mixture of xanthan gum, lecithin, sclerotium gum, pullulan, or a combination thereof in an amount less than 2% w/w, 5% w/w, or 10% w/w of the formulation. In some embodiments, the formulation comprises Siligel™ in an amount between about 1-5 w/w or 5-15% w/w, or an equivalent mixture of xanthan gum, lecithin, sclerotium gum, and pullulan. In some embodiments, the penetrant portion comprises a mixture of caprylic triglycerides and capric triglycerides in amount less than 2% w/w, 8% w/w, or 10% w/w of the formulation. In some embodiments, the formulation comprises Myritol® 312 in an amount between about 0.5-10% w/w, or an equivalent mixture of caprylic triglycerides and capric triglycerides.

In some embodiments, the penetrant portion comprises phosphatidyl choline in amount less than 12% w/w or 18% w/w of the formulation. In some embodiments, the penetrant portion comprises a phospholipid in amount less than 12% w/w or 18% w/w of the formulation. In some embodiments, the penetrant portion comprises a mixture of tridecane and undecane in amount less than 2% w/w, 5% w/w, or 8% w/w of the formulation. In some embodiments, the formulation comprises Cetiol Ultimate® in an amount less than about 2% w/w, 5% w/w, or 10% w/w, or an equivalent mixture of tridecane and undecane. In some embodiments, the penetrant portion comprises cetyl alcohol in amount less than 2% w/w, 5% w/w, or 8% w/w of the formulation. In some embodiments, the penetrant portion comprises benzyl alcohol in an amount less than about 2% w/w, 5% w/w, or 8% w/w. In some embodiments, the penetrant portion comprises stearic acid in an amount less than 2% w/w, 5% w/w, or 8% w/w of the formulation.

Lecithin organogels may be in the form of vesicles, microemulsions and micellar systems. In the form of self-assembled structures, such as vesicles or micelles, they can fuse with the lipid bilayers of the stratum corneum, thereby enhancing partitioning of encapsulated drug, as well as a disruption of the ordered bilayers structure. An example of a phospholipid-based permeation enhancement agent comprises a micro-emulsion-based organic gel defined as a semi-solid formation having an external solvent phase immobilized within the spaces available of a three-dimensional networked structure. This micro-emulsion-based organic gel in liquid phase is characterized by 1,2-diacyl-sn-glycero-3-phosphatidyl choline, and an organic solvent, which is at least one of: ethyl laureate, ethyl myristate, isopropyl myristate, isopropyl palmitate; cyclopentane, cyclooctane, trans-decalin, trans-pinane, n-pentane, n-hexane, n-hexadecane, and tripropylamine

The lecithin organogels are formulated with an additional component to assist in the formation of micelles or vascular structures. In one approach, the organogels are formulated with a polar component such as water, glycerol, ethyleneglycol or formamide, in particular with water. In general, a nonionic detergent such as a poloxamer in aqueous solution is used to top off. Alternatively, an anhydrous composition may be obtained by using, instead of a polar component, a material such as a bile salt. When formulated with bile salts, the mi cellular nature of the composition is altered so that rather than a more or less spherical vesicular form, the vesicles become wormlike and are able to accommodate larger guest molecules, as well as penetrate the epidermis more effectively. Suitable bile salts include salts of deoxycholic acid, taurocholic acid, glycocholic acid, taurochenodeoxycholic acid, glycochenodeoxycholic acid, cholic acid and the like. Certain detergents, such as Tween® 80 or Span® 80 may be used as alternatives. The percentage of these components in the anhydrous forms of the composition is in the range of 1% w/w-15% w/w. In some embodiments, the range of bile salt content is 2%-6% w/w or 1%-3.5% w/w. In these essentially anhydrous forms, powdered or micronized nonionic detergent is used to top off, typically in amounts of 20%-60% w/w. In one approach to determine the amount of bile salt, the% is calculated by dividing the % w/w of lecithin by 10.

An additional component in the formulations of the disclosure is an alcohol. Benzyl alcohol and ethanol are illustrated in the Examples. in particular, derivatives of benzyl alcohol which contain substituents on the benzene ring, such as halo, alkyl and the like. The weight percentage of benzyl or other related alcohol in the final composition is 0.5-20% w/w, and again, intervening percentages such as 1% w/w, 2% w/w, 5% w/w, 7% w/w, 10% w/w, and other intermediate weight percentages are incl tided. Due to the aromatic group present in a permeation enhancement formulation such as benzyl alcohol, the molecule has a polar end (the alcohol end) and a non-polar end (the benzene end). This enables the agent to dissolve a wider variety of drugs and agents. The alcohol concentration is substantially lower than the concentration of the lecithin organogel in the composition.

In some embodiments, as noted above, the performance of the formulations is further improved by including a nonionic detergent and polar gelling agent or including bile salts and a powdered surfactant. In both aqueous and anhydrous forms of the composition, detergents, typically nonionic detergents are added. In general, the nonionic detergent should be present in an amount of at least 2% w/w to 60% w/w. Typically, in the compositions wherein the formulation is topped off with a polar or aqueous solution containing detergent, the amount of detergent is relatively low—e.g., 2%-25% w/w, or 5-15% w/w or 7-12% w/w. However, in compositions comprising bile salts that are essentially anhydrous and are topped-off by powdered detergent, relatively higher percentages are usually used—e.g., 20%-60% w/w.

In some embodiments, the nonionic detergent provides suitable handling properties whereby the formulations are gel-like or creams at room temperature. To exert this effect, the detergent, typically a poloxamer, is present in an amount between about 2-12% w/w, preferably between about 5-25% w/w in polar formulations. In the anhydrous forms of the compositions, the detergent is added in powdered or micronized form to bring the composition to 100% and higher amounts are used. In compositions with polar constituents, rather than bile salts, the nonionic detergent is added as a solution to bring the composition to 100%. If smaller amounts of detergent solutions are needed due to high levels of the remaining components, more concentrated solutions of the nonionic detergent are employed. Thus, for example, the percent detergent in the solution may be 10% to 40% or 20% or 30% and intermediate values depending on the percentages of the other components.

Suitable nonionic detergents include poloxamers such as Poloxamer 407 (e.g. Pluronic®) and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohols and copolymers of hydrophilic and hydrophobic monomers where blocks of hydrophilic and hydrophobic portions are used.

In some embodiments, the formulation also contains surfactant, typically, nonionic surfactant at 2-25% w/w along with a polar solvent wherein the polar solvent is present in an amount at least in molar excess of the nonionic surfactant. In these embodiments, typically, the composition comprises the above-referenced amounts of lecithin organogel and benzyl alcohol along with a carbonate salt with a sufficient amount of a polar solution, typically an aqueous solution or polyethylene glycol solution that itself contains 10%-40% of surfactant, typically nonionic surfactant to bring the composition to 100%.

Other examples of surfactants include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® F127, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by Sigma-Aldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate 80 (polyoxyethylene (20) sorbitan monooleate) sold as Tween® 80, and polyoxyethylenesorbitan trioleate sold as Tween® 85 by Sigma-Aldrich. The weight percentage range of nonionic surfactant is in the range of 3% w/w-15% w/w, and again includes intermediate percentages such as 5% w/w, 7% w/w, 10% w/w, 12% w/w, and the like. In some embodiments, the detergent portion comprises a nonionic surfactant in an amount between about 2-25% w/w of the formulation; and a polar solvent in an amount less than 5% w/w of the formulation. In some embodiments, the nonionic surfactant is a poloxamer and the polar solvent is water, an alcohol, or a combination thereof. In some embodiments, the detergent portion comprises poloxamer, propylene glycol, glycerin, ethanol, 50% w/v sodium hydroxide solution, or a combination thereof. In some embodiments, the detergent portion comprises glycerin in an amount less than 3% w/w of the formulation.

In the presence of a polar gelling agent, such as water, glycerol, ethyleneglycol or formamide, a micellular structure is also often achieved. Typically, the polar agent is in molar excess of the nonionic detergent. The inclusion of the nonionic detergent/polar gelling agent combination results in a more viscous and cream-like or gel-like formulation which is suitable for application directly to the skin. This is typical of the aqueous forms of the composition.

In some embodiments other additives are included such as a gelling agent, a dispersing agent and a preservative. An example of a suitable gelling agent is hydroxypropylcellulose, which is generally available in grades from viscosities of from about 5 cps to about 25,000 cps such as about 1500 cps. All viscosity measurements are assumed to be made at room temperature unless otherwise stated. The concentration of hydroxypropylcellulose may range from about I% w/w to about 2% w/w of the composition. Other gelling agents are known in the art and can be used in place of, or in addition to hydroxypropylcellulose. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Typical components that may also be included in the formulations are fatty acids, terpenes, lipids, and cationic, and anionic detergents. In some embodiments, the formulation further comprises tranexamic acid in an amount less than 2% w/w, 5% w/w, or 10% w/w of the formulation. In some embodiments, the formulation further comprises a polar solvent in an amount less than 2% w/w, 5% w/w, 10% w/w, or 20% w/w of the formulation. In some embodiments, the formulation further comprises a humectant, an emulsifier, an emollient, or a combination thereof. In some embodiments, the formulation further comprises ethylene glycol tetraacetic acid in an amount less than about 2% w/w, 5% w/w, or 10% w/w. In some embodiments, the formulation further comprises almond oil in an amount less than about 5% w/w. In some embodiments, the formulation further comprises a mixture of thermoplastic polyurethane and polycarbonate in an amount less than about 5% w/w. In some embodiments, the formulation further comprises phosphatidylethanolamine in an amount less than about 5 w/w. In some embodiments, the formulation further comprises an inositol phosphatide in an amount less than about 5% w/w.

Other solvents and related compounds that may be used in some embodiments include acetamide and derivatives, acetone, n-alkanes (chain length between 7 and 16), alkanols, diols, short chain fatty acids, cyclohexyl-1,1-dimethylethanol, dimethyl acetamide, dimethyl formamide, ethanol, ethanol/d-limonene combination, 2-ethyl-1,3-hexanediol, ethoxydiglycol (Transcutol® by Gattefosse, Lyon, France), glycerol, glycols, lauryl chloride, limonene N-methylformamide, 2-phenylethanol, 3-phenyl-1-propanol, 3-phenyl-2-propen-1-ol, polyethylene glycol, polyoxyethylene sorbitan monoesters, polypropylene glycol 425, primary alcohols (tridecanol), 1,2-propane diol, butanediol, C₃-C₆ triols or their mixtures and a polar lipid compound selected from C₁₆ or C₁₈ monounsaturated alcohol, C₁₆ or C₁₈ branched saturated alcohol and their mixtures, propylene glycol, sorbitan monolaurate sold as Span® 20 by Sigma-Aldrich, squalene, triacetin, trichloroethanol, trifluoroethanol, trimethylene glycol and xylene.

Fatty alcohols, fatty acids, fatty esters, are bilayer fluidizers that may be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol. Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monolaurate, sorbitan trilaurate, sorbitan trioleate, sucrose coconut fatty ester mixtures, sucrose monolaurate, sucrose monooleate, tetradecyl N.N-dimethylamino acetate. Examples of suitable fatty acid. include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid. Examples of suitable fatty alcohol ethers include a-monoglyceryl ether, E0-2-oleyl ether, E0-5-oleyl ether, E0-10-oleyl ether, ether derivatives of polyglycerols and alcohols, and (1-O-dodecyl-3-O-methyl-2-O-(2′,3′-dihydroxypropyl glycerol).

Examples of completing agents that may be used in some embodiments include β- and γ-cyclodextrin complexes, hydroxypropyl methylcellulose (e.g., Carbopol® 934), liposomes, naphthalene diamide diimide, and naphthalene diester diimide.

One or more anti-oxidants may be included, such as vitamin C, vitamin E, proanthocyanidin and a-lipoic acid typically in concentrations of 0.1%-2.5% w/w.

In some particular embodiments it is desirable to adjust the pH of the formulation and the pH is adjusted to a level of pH 9-11 or 10-11, which can be done by providing appropriate buffers or simply adjusting the pH with base. In other embodiments, it is desirable to adjust the pH of the formulation to a level of pH 4-6, which can be done by providing appropriate buffers or simply adjusting the pH with an acid.

In some applications a formulation for transdermal delivery may, for example, comprise: Aveeno®, for example in an amount between about 10-95% w/w; between about 20-85% w/w, between about 20-75% w/w, between about 20-50% w/w.

In another aspect, certain embodiments are directed to a sustained release drug delivery platform releases a therapeutic compound or compounds disclosed and made as a formulation described herein over a period of, without limitation, about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In other aspects of this embodiment, a sustained release drug delivery platform releases a therapeutic compound or compounds disclosed herein with substantially first order release kinetics over a period of, without limitation, at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

In certain embodiments, kits can comprise, without limitation, one or more cream or lotion comprising one or more formulations described herein. In various embodiments, the kit can comprise formulation components for transdermal, topical, or subcutaneous administration, formulated to be administered as an emulsion coated patch. In all of these embodiments and others, the kits can contain one or more lotion, cream, patch, or the like in accordance with any of the foregoing, wherein each patch contains a single unit dose for administration to a subject.

Imaging components can optionally be included and the packaging also can include written or web-accessible instructions for using the formulation. A container can include, for example, a vial, bottle, patch, syringe, pre-filled syringe, tube or any of a variety of formats well known in the art for multi-dispenser packaging.

### Administration and Dosing

The formulations provided herein can be topically administered in any form. For administration for the treatment of skin conditions a sufficient amount of the topical composition can be applied onto a desired area and surrounding skin, for example, in an amount sufficient to cover a desired skin surface. The formulations can be applied to any skin surface, including for example, facial skin, and the skin of the hands, neck, chest and/or scalp.

In applying the formulations of the invention, the formulation itself is simply placed on the skin and spread across the surface and/or massaged to aid in penetration. The amount of formulation used is typically sufficient to cover a desired surface area. In some embodiments, a protective cover is placed over the formulation once it is applied and left in place for a suitable amount of time, i.e., 5 minutes, 10 minutes, 20 minutes or more; in some embodiments an hour or two. The protective cover can simply be a bandage including a bandage supplied with a cover that is impermeable to moisture. This essentially locks in the contact of the formulation to the skin and prevents distortion of the formulation by evaporation in some cases. The composition may be applied to the skin using standard procedures for application such as a brush, a syringe, a gauze pad, a dropper, or any convenient applicator. More complex application methods, including the use of delivery devices, may also be used, but are not required. In an alternative to administering topically to intact skin, the surface of the skin may also be disrupted mechanically by the use of spring systems, laser powered systems, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed formulations that penetrate intact skin may be applied using occlusive patches, such as those in the form micro-patches. External reservoirs of the formulations for extended administration may also be employed.

In an alternative to administering topically to intact skin, the surface of the skin may also be disrupted mechanically by the use of spring systems, laser powered systems, use of iontophoresis, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed formulations that penetrate intact skin may be applied using occlusive patches, such as those in the form micro-patches. External reservoirs of the formulations for extended administration may also be employed.

Accordingly, in certain embodiments alternative methods of administering one or more buffering agent, therapeutic compounds, agents, drugs through intact skin are provided. As nonlimiting examples, these alternative methods might be selected from the following lists: on basis of working mechanism, spring systems, laser powered, energy-propelled, Lorentz force, gas/air propelled, shock wave (including ultrasound), on basis of type of load, liquid, powder, projectile, on basis of drug delivery mechanism, nano-patches, sandpaper (microdermabrasion), iontophoresis enabled, microneedles, on basis of site of delivery, intradermal, intramuscular, and subcutaneous injection. Other suitable delivery mechanisms include, without limitation, microneedle drug delivery, such as 3M Systems, Glide SDI (pushes drug as opposed to “firing” drug), MIT low pressure injectors, micropatches (single use particle insertion device), microelectro mechanical systems (MEMS), dermoelectroporation devices (DEP), transderm ionto system (DEP), TTS transdermal therapeutic systems, membrane-moderated systems (drug reservoir totally encapsulated in a shallow compartment), adhesive diffusion-controlled system (drug reservoir in a compartment fabricated from drug-impermable metallic plastic backing), matrix dispersion type system (drug reservoir formed by homogeneously dispersing drug solids in a hydrophilic or lipophilic polymer matrix molder into medicated disc), and microreservoir system (combination of reservoir and matrix dispersion-type drug delivery system).

It has been found, generally, that the requirements for effective penetration of the skin in the case of buffers as active agents are less restrictive than those required for alternative agents useful in preventing cancer metastasis. In addition, although for these indications' delivery to the locus of the solid tumor, including melanoma, or melasma or gout is desirable, effective systemic pH alteration can be used as a way to diagnose the effectiveness of penetration when topical administration is employed.

The application method is determined by the nature of the treatment but may be less critical than the nature of the formulation itself If the application is to a skin area, it may be helpful in some instances to prepare the skin by cleansing or exfoliation. In some instances, it is helpful to adjust the pH of the skin area prior to application of the formulation itself. The application of the formulation may be by simple massaging onto the skin or by use of devices such as syringes or pumps. Patches could also be used. In some cases, it is helpful to cover the area of application to prevent evaporation or loss of the formulation.

Where the application area is essentially skin, it is helpful to seal-off the area of application subsequent to supplying the formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

In some embodiments, the disclosure is directed to administering a therapeutic agent in combination with a formulation or method provided herein. A wide variety of therapeutic agents may be used in the formulations or compositions and formulations for other routes of administration, including anesthetics, fat removal compounds, nutrients, nonsteroidal anti-inflammatory drugs (NSAIDs) agents for the treatment of migraine, hair growth modulators, antifungal agents, anti-viral agents, vaccine components, tissue volume enhancing compounds, anti-cellulite therapeutics, wound healing compounds, compounds useful to effect smoking cessation, agents for prevention of collagen shrinkage, wrinkle relief compounds such as Botox®, skin-lightening compounds, compounds for relief of bruising, cannabinoids including cannabidiols for the treatment of epilepsy, compounds for adipolysis, compounds for the treatment of hyperhidrosis, acne therapeutics, pigments for skin coloration for medical or cosmetic tattooing, sunscreen compounds, hormones, insulin, corn/callous removers, wart removers, and generally any therapeutic or prophylactic agent for which transdermal delivery is desired. As noted above, the delivery may simply affect transport across the skin into a localized subdermal location, such as treatment of nail fungus or modulation of hair growth or may affect systemic delivery such as is desirable in some instances where vaccines are used.

In addition to the compositions and formulations of the invention per se, the methods may employ a subsequent treatment with linoleic acid. As transdermal treatments generally open up the skin barrier, which is, indeed, their purpose, it is useful to seal the area of application after the treatment is finished. Thus, treatment with the formulation may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function is to allow carbonates to pass through the epidermis to the dermis at least, and, if systemic administration is achieved, through the dermis itself.

Additional therapeutic agents may be included in the compositions. For example, hydrocortisone or hydrocortisone acetate may be included in an amount ranging from 0.25% w/w to about 0.5% w/w. Menthol, phenol, and terpenoids, e.g., camphor, can be incorporated for cooling pain relief. For example, menthol may be included in an amount ranging from about 0.1% w/w to about 1.0% w/w.

The formulations can be applied in a single, one-time application, once a week, once a bi-week, once a month, or from one to twelve times daily, for a period of time sufficient to alleviate a condition, disease, disorder, symptoms, for example, for a period of time of one week, from 1 to 12 weeks or more, from 1 to 6 weeks, from 2 to 12 weeks, from 2 to 12 weeks, from 2 to 8 weeks, from 2 to 6 weeks, from 2 to 4 weeks, from 4 to 12 weeks, from 4 to 8 weeks, or from 4 to 6 weeks. The present compositions can be administered, for example, at a frequency of once per day to hourly if needed. The presently described formulations can be topically administered once or more per day for a period of time from 1 week to 4 weeks, of from 1 week to 2 weeks, for 1 week, for 2 weeks, for 3 weeks, for 4 weeks, or for 4 weeks or more. In some instances, it may also be desirable to continue treatment indefinitely for example to inhibit or prevent carcinogenesis or for improving, extending the duration of remission, or maintaining remission of a cancer or another disease or disorder. A suitable administration for a formulation comprising a skin cream, lotion or ointment, for example is once, twice, three, four times daily, or hourly if needed.

As described above, if desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A formulation in accordance with the subject matter described herein may be a topical dosage form packaged in, for example, a multi-use or single-use package, including for example, a tube, a tottle, a pump, a container or bottle, a vial, a jar, a packet, or a blister package.

Single dosage kits and packages containing a once per day amount of the topical formulation may be prepared. Single dose, unit dose, and once-daily disposable containers of the topical formulation are also provided.

The present topical formulation remains stable in storage for periods including up to about 5 years, between about 3 months and about 5 years, between about 3 months and about 4 years, between about 3 months and about 3 years, and alternately any time period between about 6 months and about 3 years.

A topical formulation described herein remains stable for up to at least 3 years at a temperature of less than or equal to 40° C. In an embodiment, the presently described topical formulation remains stable for at least 2 years at a temperature of less than or equal to 40° C. In an embodiment, the presently described formulation or emulsion remains stable for at least 3 years at a temperature of less than or equal to 40° C. and at a humidity of up to 75% RH, for at least 2 years at a temperature of less than or equal to 40° C. and at a humidity of up to 75% RH, or for at least 3 years at a temperature of less than or equal to 30° C. and at a humidity of up to 75% RH. In a further embodiment, the presently described biocompatible composition in accordance with the subject matter described herein remains stable for an extended period of time when packaged in a multi-use container such as a bottle dispenser or the like, and exhibits equal to or even greater stability when packaged in a single-use package.

In another aspect, the pharmaceutical composition of certain embodiments comprises a daily dose of particular buffering compound (e.g. sodium bicarbonate, sodium carbonate, magnesium carbonate, potassium carbonate, potassium bicarbonate, TRIS, Lysine, IEPA, etc.). A daily dose for topical or transdermal administration of any one particular buffering compound depends on the compound and animal and may be easily determined by the skilled artisan, a suitable amount is about 1 mg/kg to about 5 g/kg, and more typically the daily dose is about 10 mg/kg to about 5 g/kg, about 25 mg/kg to about 2000 mg/kg, about 50 mg/kg to about 2000 mg/kg, about 25 mg/kg to about 1000 mg/kg, about 50 mg/kg to about 1000 mg/kg, about 100 mg/kg to about 700 mg/kg, about 100 mg/kg to about 500 mg/kg, about 150 mg/kg to about 500 mg/kg, about 150 mg/kg to about 400 mg/kg, about 200 mg/kg to about 500 mg/kg, about 200 mg/kg to about 450 mg/kg, about 200 mg/kg to about 400 mg/kg, about 250 mg/kg to about 450 mg/kg, about 250 mg/kg to about 400 mg/kg, about 250 mg/kg to about 350 mg/kg, and about 275 mg/kg to about 325 mg/kg.

Alternatively, a suitable daily dose for topical or transdermal administration of each of one or more particular buffering compound (e.g. sodium bicarbonate, sodium carbonate, magnesium carbonate, potassium carbonate, potassium bicarbonate, TRIS, Lysine, IEPA, etc.) is at least about 1 mg/kg, at least about 10 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 90 mg/kg, at least about 100 mg/kg, at least about 125 mg/kg, at least about 150 mg/kg, at least about 160 mg/kg, at least about 170 mg/kg, at least about 175 mg/kg, at least about 180 mg/kg, at least about 190 mg/kg, at least about 200 mg/kg, at least about 225 mg/kg, at least about 250 mg/kg, at least about 275 mg/kg, at least about 300 mg/kg, at least about 325 mg/kg, at least about 350 mg/kg, at least about 375 mg/kg, at least about 400 mg/kg, at least about 425 mg/kg, at least about 450 mg/kg, at least about 475 mg/kg, at least about 500 mg/kg, at least about 550 mg/kg, at least about 600 mg/kg, at least about 700 mg/kg, at least about 800 mg/kg, at least about 900 mg/kg, at least about lg/kg, at least about 2 g/kg, at least about 3 g/kg, or at least about 5 g/kg.

Alternatively, a suitable dose for topical or transdermal administration of each of one or more particular buffering compound (e.g. sodium bicarbonate, sodium carbonate, magnesium carbonate, potassium carbonate, potassium bicarbonate, TRIS, Lysine, IEPA, etc.) for subject is at least about 100 mg, at least about 500 mg, at least about lg, at least about 5 g, at least about 10 g, at least about 15 g, at least about 16 g, at least about 17 g, at least about 18 g, at least about 19 g, at least about 20 g, at least about 21 g, at least about 22 g, at least about 23 g, at least about 24 g, at least about 25 g, at least about 26 g, at least about 27 g, at least about 28 g, at least about 29 g, at least about 30 g, at least about 35 g, at least about 40 g, at least about 45 g, at least about 50 g, at least about 60 g, at least about 75 g, at least about 100 g, at least about 200 g, at least about 500 g, or at least about 1.0 kg. This does may be administered daily, twice a day, three times a day, four times a day, five times a day, or more than five times a day.

Aspects of the present specification disclose that the symptoms associated with a disease or disorder described herein are reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity associated with a disease or disorder described herein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the symptoms associated with disease or disorder are reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

The formulations as described herein can be used in the manufacture of medicaments and for the treatment of humans and other animals by administration in accordance with conventional procedures.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A formulation for transdermal delivery through the skin of a subject comprising a counter ion free buffering compound, wherein the buffering compound is present in an effective amount to ameliorate or counteract an electrolyte or ion imbalance in a subject.

2. A formulation for transdermal delivery through the skin of a subject comprising one or more buffering compound, wherein at least one buffering compound has a counterion that is a cation and the one or more buffering compounds are present in a ratio and amount effective to ameliorate or counteract an electrolyte or ion imbalance in the subject.

3. A formulation according to claim 1, wherein said buffering compound is selected from Lysine (free base), TRIS, and IEPA.

4. A formulation according to claim 2, wherein the one or more buffering compound is selected from potassium bicarbonate, sodium bicarbonate, calcium carbonate, magnesium carbonate, and potassium carbonate.

5. A formulation according to claim 2, comprising potassium bicarbonate in an amount of between about 30% to about 40%, magnesium carbonate in an amount of between about 35% to about 50%, and sodium bicarbonate in an amount of between about 15% and up to about 30%, relative to each of the others w/w.

6. A formulation according to claim 2, comprising calcium carbonate in an amount of about 85% and potassium carbonate in an amount of about 15%, relative to each other w/w.

7. A formulation according to claim 2, comprising sodium bicarbonate and potassium bicarbonate in equal amounts.

8. A formulation according to claim 1, comprising TRIS base in an amount of about 20.0% w/w to about 40.0% w/w; and optionally one or more of the following:
menthol in an amount of about 0.20% w/w to about 1.0% w/w;
cetyl alcohol in an amount of about 1.0% w/w to about 5.0% w/w;
almond oil in an amount of about 1.0% w/w to about 5.0% w/w;
lipmax® in an amount of about 10.0% w/w to about 25.0% w/w;
benzyl alcohol in an amount of about 0.5% w/w to about 5.0% w/w;
poloxamer 407 (Pluronic®) in an amount of about 3.0% w/w to about 20.0% w/w;
glycerine in an amount of about 0.2% w/w to about 1.0% w/w;
propylene glycol in an amount of about 1.0% w/w to about 5.0% w/w;
NaOH (50% solution) in an amount of about 0.1% w/w to about 5.0% w/w;
ethanol in an amount of about 0.5% w/w to about 3.0% w/w; and
water in an amount to complete.

9. A formulation according to claim 1, comprising Lysine (free base) in an amount of about 20.0% w/w to about 40.0% w/w; and optionally one or more of the following:
menthol in an amount of about 0.20% w/w to about 1.0% w/w;
cetyl alcohol in an amount of about 1.0% w/w to about 5.0% w/w;
almond oil in an amount of about 1.0% w/w to about 5.0% w/w;
lipmax® in an amount of about 10.0% w/w to about 25.0% w/w;
benzyl alcohol in an amount of about 0.5% w/w to about 5.0% w/w;
poloxamer 407 (Pluronic®) in an amount of about 3.0% w/w to about 20.0% w/w;
glycerine in an amount of about 0.2% w/w to about 1.0% w/w;
propylene glycol in an amount of about 1.0% w/w to about 5.0% w/w;
NaOH (50% solution) in an amount of about 0.1% w/w to about 5.0% w/w;
ethanol in an amount of about 0.5% w/w to about 3.0% w/w; and
water in an amount to complete.

10. A formulation according to claim 1, comprising 2-imidazole-1-yl-3-ethoxycarbonylpropionic acid (IEPA) in an amount of about 20.0% w/w to about 40.0% w/w; and optionally one or more of the following:
menthol in an amount of about 0.20% w/w to about 1.0% w/w;
cetyl alcohol in an amount of about 1.0% w/w to about 5.0% w/w;
almond oil in an amount of about 1.0% w/w to about 5.0% w/w;
lipmax® in an amount of about 10.0% w/w to about 25.0% w/w;
benzyl alcohol in an amount of about 0.5% w/w to about 5.0% w/w;
poloxamer 407 (Pluronic®) in an amount of about 3.0% w/w to about 20.0% w/w;
glycerine in an amount of about 0.2% w/w to about 1.0% w/w;
propylene glycol in an amount of about 1.0% w/w to about 5.0% w/w;
NaOH (50% solution) in an amount of about 0.1% w/w to about 5.0% w/w;
ethanol in an amount of about 0.5% w/w to about 3.0% w/w; and
water in an amount to complete.

11. A formulation according to claim 2, comprising calcium carbonate in an amount of about 10.0% w/w to about 40.0% w/w; and optionally one or more of the following:
menthol in an amount of about 0.10% w/w to about 5.0% w/w;
cetyl alcohol in an amount of about 0.5% w/w to about 5.0% w/w;
almond oil in an amount of about 0.5% w/w to about 5.0% w/w;
lipmax® in an amount of about 5.0% w/w to about 30.0% w/w;
benzyl alcohol in an amount of about 0.1% w/w to about 5.0% w/w;
poloxamer 407 (Pluronic®) in an amount of about 3.0% w/w to about 20.0% w/w;
glycerine in an amount of about 0.1% w/w to about 2.0% w/w;
propylene glycol in an amount of about 0.5% w/w to about 10.0% w/w;
NaOH (50% solution) in an amount of about 0.1% w/w to about 5.0% w/w;
ethanol in an amount of about 0.3% w/w to about 5.0% w/w; and
water in an amount to complete.

12. A formulation according to claim 2, comprising:
calcium carbonate in an amount of about 5.0% w/w to about 30.0% w/w;
sodium bicarbonate in an amount of about 5.0% w/w to about 30.0% w/w;
and optionally one or more of the following:
menthol in an amount of about 0.20% w/w to about 1.0% w/w;
cetyl alcohol in an amount of about 1.0% w/w to about 5.0% w/w;
almond oil in an amount of about 1.0% w/w to about 5.0% w/w;
lipmax® in an amount of about 10.0% w/w to about 30.0% w/w;
benzyl alcohol in an amount of about 0.5% w/w to about 5.0% w/w;
poloxamer 407 (Pluronic®) in an amount of about 3.0% w/w to about 20.0% w/w;
glycerine in an amount of about 0.2% w/w to about 1.0% w/w;
propylene glycol in an amount of about 1.0% w/w to about 5.0% w/w;
NaOH (50% solution) in an amount of about 0.1% w/w to about 5.0% w/w;
ethanol in an amount of about 0.5% w/w to about 3.0% w/w; and
water in an amount to complete.

13. A method of treating an electrolyte imbalance in a patient in need thereof, the method comprising administering a formulation according to any one of claims 1-9 transdermally in an amount effective to treat the electrolyte imbalance.

14. A method of treating a cation overload or imbalance in a patient in need thereof, the method comprising administering a formulation according to any one of claims 1-9 transdermally in an amount effective to treat the cation overload or imbalance.

15. A method of treating a cation overload or imbalance in a patient caused by another transdermally administered formulation, the method comprising administering a formulation according to any one of claims 1-9 transdermally in an amount effective to treat the cation overload or imbalance.

16. A method of treating an oncology patient having a tumor lysis syndrome (TLS), the method comprising administering a formulation according to any one of claims 1-9 transdermally in an amount effective to treat or inhibit the tumor lysis syndrome.

17. A method of treating a patient having hypercalcemia, the method comprising administering a formulation according to any one of claims 1-9 transdermally in an amount effective to treat or inhibit the hypercalcemia.

18. A method of treating a patient having hypernatremia, the method comprising administering a formulation according to any one of claims 1-9 transdermally in an amount effective to treat or inhibit the hypernatremia.

19. A method of treating a patient having tumor lysis syndrome experiencing kidney failure, cardiac arrhythmias, seizures, or loss of muscle control as a result of tumor lysis syndrome, the method comprising administering a formulation according to any one of claims 1-9 transdermally in an amount effective to treat the kidney failure, cardiac arrhythmias, seizures, or loss of muscle control.

20. A method of treating a patient having abnormal levels of uric acid, the method comprising administering a formulation according to any one of claims 1-9 transdermally in an amount effective to treat or inhibit the abnormal levels of uric acid.
